(19) United States
(12) Patent Application Publication
(10) Pub. No.: US 2025/0205030 A1
(43) Pub. Date: Jun. 26, 2025

(54) EDIBLE SOFT DENTAL CHEW AND METHODS OF USING THE SAME

(71) Applicant: Alpine Pet OpCo LLC, San Francisco, CA (US)

(72) Inventors: Kerri DaSilva, Tampa, FL (US); Michael J. Stoeckle, Tampa, FL (US)

(73) Assignee: Alpine Pet OpCo LLC, San Francisco, CA (US)

(21) Appl. No.: 18/848,152

(22) PCT Filed: Mar. 20, 2023
(86) PCT No.: PCT/US2023/015599

§ 371 Date: Sep. 18, 2024

Related U.S. Application Data
(60) Provisional application No. 63/322,344, filed on Mar. 22, 2022.

(51) Int. Cl. A61D 5/00

(52) U.S. Cl. CPC A61D 5/00

(57) ABSTRACT

The present invention provides an edible soft dental chew for preventing or inhibiting dental plaque accumulation and/or dental calculus formation in a non-human animal. The edible soft dental chew includes an edible body that includes astaxanthin. The edible body has an average Shore A hardness of less than about 60. The present invention further provides a method of preventing or inhibiting dental plaque accumulation and/or dental calculus formation in a subject in need thereof.

### CROSS REFERENCE TO RELATED APPLICATION

The present application claims priority to and the benefit of U.S. Provisional Application No. 63/322,344, filed Mar. 22, 2022, the contents of which are herein incorporated by reference in their entirely.

### FIELD OF THE INVENTION

The present invention relates to an edible soft dental chew and methods for preventing or inhibiting dental plaque accumulation and/or dental calculus formation in a subject in need thereof

### BACKGROUND

Non-human animals, such as canines and felines, are susceptible to dental plaque accumulation and/or dental calculus (i.e., tartar) formation. Dental plaque is a soft deposit which forms on the surfaces of teeth. Dental plaque is generally believed to be formed as a byproduct of bacterial growth and comprises a dense microbial layer consisting of a mass of microorganisms embedded in a polysaccharide matrix. Dental plaque adheres to the surfaces of teeth, especially along irregular and rough surfaces, and is typically found at the gingival margin, in cracks in tooth enamel, and on the surface of built-up dental calculus. Dental plaque can give rise to tooth decay and periodontal problems, such as gingivitis and periodontitis.

Dental plaque formed along tooth surfaces provides a locus for dental calculus formation. Dental calculus is a hardened (i.e., mineralized) form of dental plaque resulting from precipitation of minerals from saliva and gingival crevicular fluid (GCF) in dental plaque on teeth. Continued accumulation of both dental plaque and dental calculus can result in gum recession and, ultimately, destruction of bone and periodontal ligament.

Conventional edible dental chews for non-human animals rely on mechanical removal (e.g., abrasion) to reduce levels of dental plaque and/or dental calculus. In addition, dental plaque and dental calculus may be removed via brushing. While effective at removing dental plaque and/or dental calculus that has already formed and accumulated on teeth of non-human animals, neither conventional edible dental chews nor brushing effectively prevent or inhibit dental plaque accumulation and/or dental calculus formation.

Furthermore, conventional edible dental chews also have an average Shore A hardness that is high in order to facilitate mechanical removal of dental plaque and/or dental calculus. For this reason, conventional edible dental chews may be unsuitable for non-human animals having missing teeth, deciduous teeth, cracked teeth, and/or a preference for softer foods/chews.

As such, there remains a need to provide improved edible soft dental chews and methods for preventing or inhibiting dental plaque accumulation and/or dental calculus formation in non-human animals.

### SUMMARY OF THE INVENTION

The present invention provides an edible soft dental chew for preventing or inhibiting dental plaque accumulation and/or dental calculus formation in a non-human animal. The edible soft dental chew includes an edible body that includes astaxanthin. The edible body has an average Shore A hardness of less than about 60. In some embodiments, the edible body has an average Shore A hardness of less than about 50.

In some embodiments, the edible body includes from about 0.01 to about 10 mg of astaxanthin. In some embodiments, the edible body includes from about 0.01 to about 4 mg of astaxanthin. In some embodiments the edible body comprises a hardness reducing ingredient. In some embodiments, the hardness reducing ingredient is meat. In some embodiments, the hardness reducing ingredient is beef, pork, or chicken. In some embodiments, the hardness reducing ingredient is chicken. In some embodiments, the hardness reducing ingredient is chicken meal.

In some embodiments, the edible body further includes a core and an outer layer. The outer layer is disposed on the core. At least one of the core and the outer layer include the astaxanthin. The outer layer has an average Shore A hardness of less than about 60. In some embodiments, the outer layer has an average Shore A hardness of less than about 50.

In some embodiments, the core includes one or more of a polishing agent, a flavor additive, a vegetable oil, a humectant, a dental additive, a food additive, a hardness reducing ingredient, and water. In some embodiments, the core has an average Shore A hardness of less than about 50. In some embodiments, the core has an average Shore A hardness of less than about 40.

In some embodiments, the outer layer comprises the astaxanthin. In some embodiments, the outer layer comprises from about 0.01 mg to about 10 mg of astaxanthin. In some embodiments, the outer layer comprises from about 0.2 mg to about 5 mg of astaxanthin. In some embodiments, the outer layer comprises from about 0.3 mg to about 4 mg of astaxanthin. In some embodiments, the outer layer comprises from about 0.2 mg to about 0.8 mg of astaxanthin. In some embodiments, the outer layer comprises from about 0.75 mg to about 1.75 mg of astaxanthin.

In some embodiments, the core includes a polishing agent selected from sodium bicarbonate, calcium carbonate, dicalcium phosphate, calcium sulfate, silica, iron oxide, hydroxyapatite, aluminum oxide, perlite, plastic particles, or any combination thereof. In some embodiments, the core includes a polishing agent selected from calcium carbonate, dicalcium phosphate, sodium bicarbonate, or any combination thereof.

In some embodiments, the core includes a flavor additive selected from spearmint, peppermint, wintergreen, sassafras, clove, sage, eucalyptus, marjoram, cinnamon, lemon, lime, grapefruit, coconut, orange, vanilla, rosemary, salt, chicken flavor, honey, or any combination thereof. In some embodiments, the core includes a flavor additive selected from clove, cinnamon, vanilla, rosemary, salt, chicken flavor, honey, or any combination thereof.

In some embodiments, the core includes a vegetable oil selected from palm oil, soybean oil, canola oil, sunflower seed oil, flaxseed oil, peanut oil, cottonseed oil, palm kernel oil, coconut oil, olive oil, or any combination thereof. In some embodiments, the core includes a vegetable oil selected from canola oil, flaxseed oil, or any combination thereof.

In some embodiments, the core includes a humectant selected from glycerin, sorbitol, or any combination thereof. In some embodiments, the humectant is glycerin.

In some embodiments, the core includes a dental additive selected from glucose oxidase, L-ascorbic acid phosphate, lactoperooxidase, tetrasodium pyrophosphate, or any combination thereof. In some embodiments, the core includes a dental additive selected from glucose oxidase, L-ascorbic acid phosphate, tetrasodium pyrophosphate, or any combination thereof.

In some embodiments, the core includes a food additive selected from cultured dextrose, gelatin, lecithin, rice flour, citric acid, or any combination thereof. In some embodiments, the core includes water.

In some embodiments, the outer layer includes an exterior surface facing away from the core that is substantially free of any protrusions configured to remove dental plaque and/or dental calculus from teeth of a subject.

In some embodiments, the core includes a hardness reducing ingredient. In some embodiments, the outer layer includes a hardness reducing ingredient. In some embodiments, the hardness reducing ingredient is chicken meat.

In some embodiments, the edible body is disc-shaped. In some embodiments, the edible body has a cookie shape.

Another aspect of the present invention provides a method of preventing or inhibiting dental plaque accumulation and/or dental calculus formation in a subject in need thereof. The method includes administering to the subject at least one edible soft dental chew at least once per day. The edible soft dental chew includes an edible body that includes astaxanthin. The edible body has an average Shore A hardness of less than about 60. In some implementations, the edible body has an average Shore A hardness of less than about 50. The subject is a non-human animal.

In some implementations, the edible body further includes a core and an outer layer. The outer layer is disposed on the core. At least one of the core and the outer layer includes the astaxanthin. The outer layer has an average Shore A hardness of less than about 60. In some implementations, the outer layer has an average Shore A hardness of less than about 50.

In some implementations, the core has an average Shore A hardness of less than about 50. In some implementations, the core has an average Shore A hardness of less than about 40.

In some implementations, the core includes a hardness reducing ingredient. In some implementations, the outer layer includes a hardness reducing ingredient. In some implementations, the hardness reducing ingredient is chicken meat.

In some implementations, the outer layer includes from about 0.01 mg to about 10.0 mg of astaxanthin.

In some implementations, the non-human animal has a weight of up to about 8 lbs, the outer layer includes from about 0.2 mg to about 0.8 mg of astaxanthin, and the subject is administered at least one edible soft dental chew at least once per day. In some implementations, the subject is administered one edible soft dental chew twice per day.

In some implementations, the non-human animal has a weight of up to about 15 lbs, the outer layer includes from about 0.2 mg to about 0.8 mg of astaxanthin, and the subject is administered at least one edible soft dental chew at least once per day. In some implementations, the subject is administered one edible soft dental chew twice per day.

In some implementations, the non-human animal has a weight of from about 8 lbs to about 20 lbs, the outer layer includes from about 1.8 mg to about 2 mg of astaxanthin, and the subject is administered at least one edible soft dental chew at least once per day. In some implementations, the subject is administered two edible soft dental chews twice per day.

In some implementations, the non-human animal has a weight of from about 15 lbs to about 30 lbs, the outer layer includes from about 0.2 mg to about 0.8 mg of astaxanthin, and the subject is administered at least one edible soft dental chew at least once per day. In some implementations, the subject is administered two edible soft dental chews twice per day.

In some implementations, the non-human animal has a weight of from about 20 lbs to about 50 lbs, the outer layer includes from about 2.6 mg to about 4 mg of astaxanthin, and the subject is administered at least one edible soft dental chew at least once per day. In some implementations, the subject is administered one edible soft dental chew twice per day.

In some implementations, the non-human animal has a weight of from about 30 lbs to about 50 lbs, the outer layer includes from about 0.75 mg to about 1.75 mg of astaxanthin, and the subject is administered at least one edible soft dental chew at least once per day. In some implementations, the subject is administered one edible soft dental chew twice per day.

In some implementations, the non-human animal has a weight of greater than about 40 lbs, the outer layer includes about 4 mg of astaxanthin, and the subject is administered at least one edible soft dental chew at least once per day. In some implementations, the subject is administered two edible soft dental chews twice per day.

In some implementations, the non-human animal has a weight of greater than about 50 lbs, the outer layer includes from about 0.75 to about 1.75 mg of astaxanthin, and the subject is administered at least one edible soft dental chew at least once per day. In some implementations, the subject is administered two edible soft dental chews twice per day.

In some implementations, the non-human animal is at least 12 weeks old. In some implementations, the non-human animal is a canine or a feline.

In some implementations, the non-human animal has one or more of missing teeth, deciduous teeth, and cracked teeth. In some implementations, teeth of the non-human animal are cleaned prior to the administration of the at least one edible soft dental chew.

In some implementations, teeth of the non-human animal are substantially free of dental plaque prior to the administration of the at least one edible soft dental chew. In some implementations, teeth of the non-human animal are substantially free of dental calculus prior to the administration of the at least one edible soft dental chew.

Another aspect of the present invention provides a process for preparing an edible soft dental chew for preventing or inhibiting dental plaque accumulation and/or dental calculus formation in a non-human animal. The edible soft dental chew includes an edible body that includes astaxanthin. The edible body further includes a core and an outer layer. The outer layer is disposed on the core. At least one of the core and the outer layer includes the astaxanthin. The outer layer has an average Shore A hardness of less than about 60. The process includes extruding the core. The process also includes extruding the outer layer on the core to form the edible body.

In some implementations, the step of extruding the core and the step of extruding the outer layer are performed simultaneously. In other implementations, the step of extruding the outer layer is performed after the step of extruding the core.

### BRIEF DESCRIPTION OF THE DRAWINGS

The figures below are provided by way of example and are not intended to limit the scope of the claimed invention.

FIG. 1 is a perspective view of one embodiment of an edible soft dental chew for preventing or inhibiting dental plaque accumulation and/or dental calculus formation.

FIG. 2 is a transverse cross-sectional view of the edible soft dental chew of FIG. 1.

FIG. 3 is a perspective view of another embodiment of an edible soft dental chew for preventing or inhibiting dental plaque accumulation and/or dental calculus formation.

FIG. 4 is a transverse cross-sectional view of the edible soft dental chew of FIG. 3.

### DETAILED DESCRIPTION

### I. Definitions

As used herein, the term “preventing or inhibiting” with respect to dental plaque accumulation and/or dental calculus formation refers to eliminating dental plaque accumulation and/or dental calculus formation in a subject or reducing dental plaque formation and/or dental calculus formation in a subject. It will be appreciated that dental plaque accumulation and/or dental calculus formation may be inhibited by about 10%, 15%, 20%, 25%, 30%, 35%, 40%, 45%, 50%, 55%, 60%, 65%, 70%, 75%, 80%, 85%, 90%, 95%, 96%, 97%, 97.5%, 98%, 98.5%, 99%, 99.5%, 99.9%, or 99.99% as compared to an untreated subject.

As used herein, “average Shore A hardness” refers to the Shore A hardness of an object as measured with a Shore A durometer. In some embodiments, the average Shore A hardness is measured in accordance with ASTM D2240 type A.

The dental chew comprises an edible body. In some embodiments, the edible body is chewable. In some embodiments, the edible body may be a single layer of edible material. In some embodiments, the edible body may include more than one layer of edible material. For example, the edible body may include a core and an outer layer.

As used herein, the term “core” refers to an edible material that is at least partially surrounded by another layer. In some embodiments, the core may be entirely surrounded by another layer. In some embodiments, the core is chewable.

As used herein, the term “outer layer” refers to an edible material that is disposed on the core and at least partially surrounds the core. In some embodiments, the outer layer entirely surrounds the core. In some embodiments, the outer layer is chewable. In some embodiments, one or more intermediate layers are disposed between the core and the outer layer.

As used herein, the term “about”, when referring to a numerical value or range of values, allows for a degree of variability in the value or range or values, for example, within 10%, or within 5% of a stated value or of a stated limit of a range.

As used herein, the term “subject” to which administration is contemplated includes, but is not limited to, a human and/or a non-human animal, e.g., a mammal such as primates, cattle, pigs, horses, sheep, goats, rodents, cats, and/or dogs. Preferably, the subject is a non-human animal. More preferably, the subject is a companion animal or pet. Even more preferably, the subject is a canine or a feline (e.g., a dog or a cat).

In general, the “effective amount” of an edible soft dental chew refers to an amount sufficient to elicit the desired biological response, e.g., to prevent or inhibit dental plaque accumulation and/or dental calculus formation. As will be appreciated by those of ordinary skill in the art, the effective amount of a compound may vary depending on such factors as the desired biological endpoint, the pharmacokinetics of the compound, the mode of administration, and the age, weight, health, and condition of the subject.

An effective amount can be administered in one or more administrations, applications or dosages. An effective amount can be administered from one or more times per day to one or more times per week; including once every other day. In some embodiments, the effective amount is administered at least once per day. In some embodiments, the effective amount is administered daily before or after a meal, e.g., immediately before or after a meal or 15-60 minutes before or after a meal. In some embodiments, the compositions are administered orally.

In some embodiments, the present invention contemplates administration of an effective amount of the edible soft dental chew as a prophylactic before a subject begins to suffer from dental plaque accumulation or dental calculus formation. As used herein, and unless otherwise specified, a “prophylactically effective amount” of an edible soft dental chew is an amount sufficient to prevent or inhibit dental plaque accumulation and/or dental calculus formation. A prophylactically effective amount of an edible soft dental chew means an amount of an edible soft dental chew, alone or in combination with other agents, which provides a prophylactic benefit in the prevention or inhibition of dental plaque accumulation and/or dental calculus formation. The term “prophylactically effective amount” can encompass an amount that improves overall prophylaxis or enhances the prophylactic efficacy of another prophylactic agent.

As used herein, the term “onset of administration” refers to the day on which administration of the edible soft dental chew begins.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

### II. Edible Soft Dental Chew

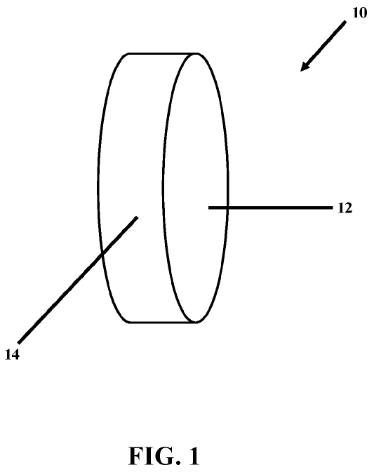

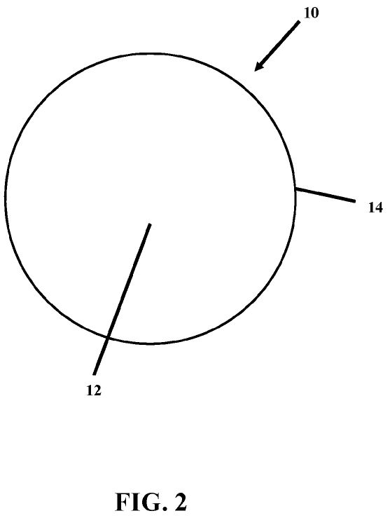

With reference to FIGS. 1 and 2, the present invention provides an edible soft dental chew 10 for preventing or inhibiting dental plaque accumulation and/or dental calculus formation in a non-human animal. The edible soft dental chew 10 includes an edible body 12. The edible body 12 includes astaxanthin. The edible body 12 has an average Shore A hardness of less than about 60.

With continued reference to FIGS. 1 and 2, the edible body 12 is an edible material. In some embodiments, the edible body 12 is chewable. As shown in FIG. 1, in some embodiments, the edible body 12 has a disc configuration. In other embodiments, the edible body 12 may have a prism configuration, a spherical configuration, a cone configuration, or a bone configuration.

The geometrical configuration of a cross-sectional shape defined by the edible body 12 is not particularly limited, and may be any cross-sectional shape suitable for consumption by a non-human animal. By way of non-limiting example, the cross-sectional shape defined by the edible body 12 may be a triangle, square, rectangle, oval, circle, square, diamond, heart, star, bone, cookie-shape or any other geometric shape. As shown in FIG. 2, in some embodiments, the cross-sectional shape defined by the edible body 12 is a circle.

In some embodiments, the edible body 12 includes from about 0.01 mg to about 10 mg of astaxanthin. In some embodiments, the edible body 12 includes from about 0.05 mg to about 10 mg of astaxanthin. In some embodiments, the edible body 12 includes from about 0.1 mg to about 0.9 mg of astaxanthin. In some embodiments, the edible body 12 includes from about 0.2 mg to about 0.8 mg of astaxanthin. In some embodiments, the edible body 12 includes from about 0.25 mg to about 0.75 mg of astaxanthin. In some embodiments, the edible body 12 includes from about 0.3 mg to about 0.7 mg of astaxanthin. In some embodiments, the edible body 12 includes from about 0.35 mg to about 0.65 mg of astaxanthin. In some embodiments, the edible body 12 includes from about 0.4 mg to about 0.6 mg of astaxanthin. In some embodiments, the edible body 12 includes from about 0.45 mg to about 0.55 mg of astaxanthin. In some embodiments, the edible body 12 includes about 0.5 mg of astaxanthin. In some embodiments, the edible body 12 includes from about 0.01 mg to about 4 mg of astaxanthin. In some embodiments, the edible body 12 includes about 0.2 mg to about 5 mg of astaxanthin. In some embodiments, the edible body 12 includes from about 0.3 mg to about 4 mg of astaxanthin.

In some embodiments, the edible body 12 includes from about 0.5 mg to about 2.0 mg of astaxanthin. In some embodiments, the edible body 12 includes from about 0.6 mg to about 1.9 mg of astaxanthin. In some embodiments, the edible body 12 includes from about 0.7 mg to about 1.8 mg of astaxanthin. In some embodiments, the edible body 12 includes from about 0.75 mg to about 1.75 mg of astaxanthin. In some embodiments, the edible body 12 includes from about 0.8 mg to about 1.7 mg of astaxanthin. In some embodiments, the edible body 12 includes from about 0.85 mg to about 1.65 mg of astaxanthin. In some embodiments, the edible body 12 includes from about 0.9 mg to about 1.6 mg of astaxanthin. In some embodiments, the edible body 12 includes from about 0.95 mg to about 1.55 mg of astaxanthin. In some embodiments, the edible body 12 includes from about 1.0 mg to about 1.5 mg of astaxanthin. In some embodiments, the edible body 12 includes from about 1.05 mg to about 1.45 mg of astaxanthin. In some embodiments, the edible body 12 includes from about 1.10 mg to about 1.40 mg of astaxanthin. In some embodiments, the edible body 12 includes from about 1.15 mg to about 1.35 mg of astaxanthin. In some embodiments, the edible body 12 includes from about 1.20 mg to about 1.30 mg of astaxanthin. In some embodiments, the edible body 12 includes about 1.25 mg of astaxanthin.

In some embodiments, the edible body 12 includes from about 0.5 mg to about 1.5 mg of astaxanthin. In some embodiments, the edible body 12 includes about 1.0 mg of astaxanthin. In some embodiments, the edible body 12 includes from about 1.5 mg to about 2.5 mg of astaxanthin. In some embodiments, the edible body 12 includes about 2.0 mg of astaxanthin. In some embodiments, the edible body 12 includes from about 2.0 mg to about 3.0 mg of astaxanthin. In some embodiments, the edible body 12 includes about 2.5 mg of astaxanthin. In some embodiments, the edible body 12 includes from about 4.5 mg to about 5.5 mg of astaxanthin. In some embodiments, the edible body 12 includes about 5.0 mg of astaxanthin.

In some embodiments, the edible body 12 has an average Shore A hardness of less than about 57.5. In some embodiments, the edible body 12 has an average Shore A hardness of less than about 55. In some embodiments, the edible body 12 has an average Shore A hardness of less than about 52.5. In some embodiments, the edible body 12 has an average Shore A hardness of less than about 50. In some embodiments, the edible body 12 has an average Shore A hardness of less than about 45. In some embodiments, the edible body 12 has an average Shore A hardness of less than about 40. In some embodiments, the edible body 12 has an average Shore A hardness of less than about 35. In some embodiments, the edible body 12 has an average Shore A hardness of less than about 30. In some embodiments, the edible body 12 has an average Shore A hardness of less than about 25. In some embodiments, the edible body 12 has an average Shore A hardness of less than about 20. In some embodiments, the edible body 12 has an average Shore A hardness of less than about 15. In some embodiments, the edible body 12 has an average Shore A hardness of less than about 10.

In some embodiments, the edible body 12 has an average Shore 00 hardness of less than about 30. In some embodiments, the edible body 12 has an average Shore 00 hardness of less than about 20. In some embodiments, the edible body 12 has an average Shore 00 hardness of less than about 15.

In some embodiments, the edible body 12 has an average Shore A hardness of from about 40 to about 59. In some embodiments, the edible body 12 has an average Shore A hardness of from about 42.5 to about 57.5. In some embodiments, the edible body 12 has an average Shore A hardness of from about 45 to about 55. In some embodiments, the edible body 12 has an average Shore A hardness of from about 47.5 to about 52.5. In some embodiments, the edible body 12 has an average Shore A hardness of about 50.

In some embodiments, the edible body 12 has an average Shore A hardness of from about 10 to about 30. In some embodiments, the edible body 12 has an average Shore A hardness of from about 10 to about 20. In some embodiments, the edible body 12 has an average Shore A hardness of from about 20 to about 30. In some embodiments, the edible body 12 has an average Shore A hardness of from about 20 to about 40. In some embodiments, the edible body 12 has an average Shore A hardness of from about 30 to about 40. In some embodiments, the edible body 12 has an average Shore A hardness of from about 30 to about 50. In some embodiments, the edible body 12 has an average Shore A hardness of from about 40 to about 50.

In some embodiments, the edible body 12 includes one or more of a polishing agent, a flavor additive, a vegetable oil, a humectant, a dental additive, a food additive, a hardness reducing ingredient, and water.

In some embodiments, the edible body 12 includes the hardness reducing ingredient. When present, the hardness reducing ingredient reduces the average Shore A hardness of the edible body 12. The hardness reducing ingredient is not particularly limited, and may be any hardness reducing ingredient suitable for consumption by a non-human animal that reduces the average Shore A hardness of the edible body 12. In some embodiments, the hardness reducing ingredient is a meat. In some embodiments, the meat is chicken, pork, or beef. In some embodiments, the hardness reducing ingredient is chicken meat. The chicken meat may be chicken meal. The chicken meat may be dehydrated chicken meat. Practically speaking, the hardness reducing ingredient may be used in place of all or a portion of other harder ingredients in the edible chew 10, such as flours (e.g., rice flour).

In some embodiments, the edible chew comprises from about 1 wt % to about 50 wt % of the hardness reducing ingredient (e.g., chicken), by percent weight of the chew. In some embodiments, the edible chew comprises from about 1 wt % to about 50 wt % of the hardness reducing ingredient (e.g., chicken), by percent weight of the chew. In some embodiments, the edible chew comprises from about 2 wt % to about 40 wt % of the hardness reducing ingredient (e.g., chicken), by percent weight of the chew. In some embodiments, the edible chew comprises from about 4 wt % to about 25 wt % of the hardness reducing ingredient (e.g., chicken), by percent weight of the chew. In some embodiments, the edible chew comprises from about 5 wt % to about 20 wt % of the hardness reducing ingredient (e.g., chicken), by percent weight of the chew. In some embodiments, the edible chew comprises from about 5 wt % to about 10 wt % of the hardness reducing ingredient (e.g., chicken), by percent weight of the chew. In some embodiments, the edible chew comprises from about 5 wt % to about 50 wt % of the hardness reducing ingredient (e.g., chicken), by percent weight of the chew. In some embodiments, the edible chew comprises from about 8 wt % to about 20 wt % of the hardness reducing ingredient (e.g., chicken), by percent weight of the chew. In some embodiments, the edible chew comprises from about 8 wt % to about 50 wt % of the hardness reducing ingredient (e.g., chicken), by percent weight of the chew.

In some embodiments, the edible body 12 includes the polishing agent. When present, the polishing agent mechanically removes (i.e., via a polishing action) dental plaque and/or dental calculus from teeth of the non-human animal. The polishing agent is not particular limited, and may be any polishing agent suitable for consumption by a non-human animal that mechanically removes dental plaque and/or dental calculus from teeth. In some embodiments, the edible body 12 includes a polishing agent selected from sodium bicarbonate, calcium carbonate, dicalcium phosphate, calcium sulfate, silica, iron oxide, hydroxyapatite, aluminum oxide, perlite, plastic particles, or any combination thereof. In some embodiments, the edible body 12 includes a polishing agent selected from calcium carbonate, dicalcium phosphate, sodium bicarbonate, or any combination thereof. In some embodiments, the polishing agent includes calcium carbonate. In some embodiments, the polishing agent includes dicalcium phosphate. In some embodiments, the polishing agent includes sodium bicarbonate. In some embodiments, the polishing agent includes calcium carbonate, dicalcium phosphate, and sodium bicarbonate. In some embodiments, the polishing agent is a combination of calcium carbonate, dicalcium phosphate, and sodium bicarbonate.

In some embodiments, the edible body 12 includes the flavor additive. When present, the flavor additive improves the palatability of the edible soft dental chew 10 and/or freshens breath of the non-human animal upon consumption of the edible soft dental chew 10. The flavor additive is not particular limited, and may be any flavor additive suitable for consumption by a non-human animal that is palatable to and/or freshens breath of the non-human animal. In some embodiments, the edible body 12 includes a flavor additive selected from spearmint, peppermint, wintergreen, sassafras, clove, sage, eucalyptus, marjoram, cinnamon, lemon, lime, grapefruit, coconut, orange, vanilla, rosemary, salt, chicken flavor, honey, or any combination thereof. In some embodiments, the edible body 12 includes a flavor additive selected from clove, cinnamon, vanilla, rosemary, salt, chicken flavor, honey, or any combination thereof. In some embodiments, the flavor additive includes clove. In some embodiments, the flavor additive includes cinnamon. In some embodiments, the flavor additive includes vanilla. In some embodiments, the flavor additive includes rosemary. In some embodiments, the flavor additive includes salt. In some embodiments, the flavor additive includes chicken flavor. In some embodiments, the flavor additive honey. In some embodiments, the flavor additive includes clove, cinnamon, vanilla, rosemary, salt, chicken flavor, and honey. In some embodiments, the flavor additive is a combination of clove, cinnamon, vanilla, rosemary, salt, chicken flavor, and honey.

In some embodiments, the edible body 12 includes the vegetable oil. The vegetable oil is not particular limited, and may by any vegetable oil suitable for consumption by a non-human animal. In some embodiments, the edible body 12 includes a vegetable oil selected from palm oil, soybean oil, canola oil, sunflower seed oil, flaxseed oil, peanut oil, cottonseed oil, palm kernel oil, coconut oil, olive oil, or any combination thereof. In some embodiments, the edible body 12 includes a vegetable oil selected from selected from canola oil, flaxseed oil, or any combination thereof. In some embodiments, the vegetable includes canola oil. In some embodiments, the vegetable oil includes flaxseed oil. In some embodiments, the vegetable oil includes canola oil and flaxseed oil. In some embodiments, the vegetable oil is a combination of canola oil and flaxseed oil.

In some embodiments, the edible body 12 includes the humectant. When present, the humectant retains moisture in the edible soft dental chew 10 and may allow for greater control over shape, viscosity, texture, and/or shelf-life of the edible soft dental chew 10. The humectant is not particular limited, and may by any humectant suitable for consumption by a non-human animal that retains moisture in the edible soft dental chew 10. In some embodiments, the edible body 12 includes a humectant selected from sucrose, glycerin, glycerol, sorbitol, xylitol, butylene glycol, propylene glycol, or any combination thereof. In some embodiments, the edible body 12 includes a humectant selected from glycerin, sorbitol, or any combination thereof. In some embodiments, the humectant includes glycerin. In some embodiments, the humectant includes sorbitol. In some embodiments, the humectant includes glycerin and sorbitol. In some embodiments, the humectant is glycerin.

In some embodiments, the edible body 12 includes the dental additive. When present, the dental additive contributes to oral health of teeth of the non-human animal (e.g., anti-dental plaque agents, anti-dental calculus agents, etc.) and/or visual appearance (e.g., whitening) of teeth of the non-human animal. The dental additive is not particularly limited, and may by any dental additive suitable for consumption by a non-human animal that contributes to oral health and/or visual appearance of teeth of the non-human animal. By way of non-limiting example, the dental additive may be one or more of an anti-dental plaque agent, an anti-dental calculus agent, and a whitening agent. In some embodiments, the edible body 12 includes a dental additive selected from glucose oxidase, L-ascorbic acid phosphate, lactoperooxidase, tetrasodium pyrophosphate, or any combination thereof. In some embodiments, the dental additive includes glucose oxidase. In some embodiments, the dental additive includes L-ascorbic acid phosphate. In some embodiments, the dental additive includes lactoperooxidase. In some embodiments, the dental additive includes tetrasodium pyrophosphate. In some embodiments, the dental additive includes glucose oxidase, L-ascorbic acid phosphate, and tetrasodium pyrophosphate. In some embodiments, the dental additive is a combination of glucose oxidase, L-ascorbic acid phosphate, and tetrasodium pyrophosphate.

In some embodiments, the edible body 12 includes the food additive. When present, the food additive preserves flavor and/or enhances taste, appearance, and/or texture of the edible soft dental chew 10. The food additive is not particular limited, and may be any food additive suitable for consumption by a non-human animal that preserves flavor and/or enhances taste, appearance, and/or texture of the edible soft dental chew 10. By way of non-limiting example, the food additive may be one or more of an acidulant, an acidity regulator, an anticaking agent, an antifoaming agent, a foaming agent, a bulking agent, an antioxidant, a food coloring, a fortifying agent, a color retention agent, an emulsifier, a flour treatment agent, a glazing agent, a preservative, a stabilizer, and a thickener. In some embodiments, the edible body 12 includes a food additive selected from cultured dextrose, gelatin, lecithin, rice flour, citric acid, or any combination thereof. In some embodiments, the food additive includes cultured dextrose. In some embodiments, the food additive includes gelatin. In some embodiments, the food additive includes rice flour. In some embodiments, the food additive includes citric acid. In some embodiments, the food additive includes cultured dextrose, gelatin, lecithin, rice flour, and citric acid. In some embodiments, the food additive is a combination of cultured dextrose, gelatin, lecithin, rice flour, and citric acid.

In some embodiments, the edible body 12 includes water. In some embodiments, the edible body 12 includes the polishing agent, the flavor additive, the vegetable oil, the humectant, the dental additive, the food additive, the hardness reducing ingredient, and water. In some embodiments, the edible body 12 includes calcium carbonate, canola oil, cinnamon, citric acid, clove, cultured dextrose, dicalcium phosphate, flaxseed oil, gelatin, glycerin, honey, L-ascorbic acid phosphate, lecithin, rice flour, salt, sodium bicarbonate, tetrasodium pyrophosphate, vanilla, chicken meat, and water.

In some embodiments, the edible body 12 includes one or more binders and/or fillers. When present, the binders and/or fillers may be any binders and/or fillers suitable for consumption by a non-human animal. In some embodiments, the edible body 12 is an extrudate.

As will be appreciated by those of ordinary skill in the art, one or more the polishing agents, the flavor additives, the vegetable oils, the humectants, the dental additives, and/or the food additives described herein may also function as another of the polishing agent, the flavor additive, the vegetable oil, the humectant, the dental additive, and/or the food additive. For example, when the flavor additive includes honey, honey may also function as a humectant. As another example, when the humectant includes sorbitol, sorbitol may also function as a flavor additive. As yet another example, when the flavor additive is salt, the salt may serve as a food additive (e.g., a preservative).

With continued reference to FIGS. 1 and 2, the edible body 12 includes an exterior surface 14. As shown in FIGS. 1 and 2, the exterior surface 14 may be substantially free of any protrusions configured to remove dental plaque and/or dental calculus from teeth of the non-human animal. In some embodiments, the exterior surface may include protrusions configured to remove dental plaque and/or dental calculus from teeth of the non-human animal. Suitable protrusions for inclusion on the exterior surface 14 of the edible body 12 are described in U.S. Patent Application Publication No. 2011/0052661, which is hereby incorporated by reference in its entirety.

Without wishing to be bound by theory, it is believed that the astaxanthin in the edible soft dental chew 10 of the present invention results in an increased concentration of salivary immunoglobulin A (sigA) in a non-human animal after consumption of the edible soft dental chew 10, thereby preventing or inhibiting dental plaque accumulation and/or dental calculus formation. Accordingly, the edible soft dental chew 10 is suitable for preventing or inhibiting dental plaque accumulation and/or dental calculus formation in non-human animals. This prevention or inhibition of dental plaque accumulation and/or dental calculus formation is achieved regardless of whether protrusions configured to remove dental plaque and/or dental calculus from teeth of the non-human animal are present on the exterior surface 14 of the edible body 12. Moreover, this prevention or inhibition of dental plaque accumulation and/or dental calculus formation is achieved regardless of the average Shore A hardness of the edible body 12. In other words, the edible soft dental chew 10 is not reliant on mechanical removal of dental plaque and/or dental calculus, either via protrusions or via hardness of the edible body 12, in order to prevent or inhibit dental plaque accumulation and/or dental calculus formation in non-human animals. As such, the edible soft dental chew 10 is suitable for administration to non-human animals having missing teeth, deciduous teeth, cracked teeth, and/or a preference for softer foods/chews.

In some embodiments, the edible soft dental chew 10 is free of, or substantially free of, antioxidants other than astaxanthin. In some embodiments, the edible soft dental chew is free of, or substantially free of Vitamin E. In some embodiments, the edible soft dental chew is free of, or substantially free of Vitamin C. In some embodiments, the ratio of astaxanthin to other antioxidants is 1:1 or greater (e.g., 2:1, 3:1, 5:1, or 10:1). In some embodiments, the ratio of astaxanthin to Vitamin C is 1:1 or greater (e.g., 2:1, 3:1, 5:1, or 10:1). In some embodiments, the ratio of astaxanthin to Vitamin E is 1:1 or greater (e.g., 2:1, 3:1, 5:1, or 10:1).

In some embodiments, the edible body 12 includes one or more of the polishing agent, the flavor additive, the vegetable oil, the humectant, the dental additive, the food additive, the hardness reducing ingredient, and water. The edible body 12 also includes from about 0.05 mg to about 10.0 mg of astaxanthin. The edible body 12 has an average Shore A hardness of less than about 60.

In some embodiments, the edible body 12 includes the hardness reducing ingredient. The edible body 12 also includes from about 0.05 mg to about 10.0 mg of astaxanthin. The edible body 12 has an average Shore A hardness of less than about 60.

In some embodiments, the edible body 12 includes the polishing agent. The edible body 12 also includes from about 0.05 mg to about 10.0 mg of astaxanthin. The polishing agent is selected from calcium carbonate, dicalcium phosphate, sodium bicarbonate, or any combination thereof. The edible body 12 has an average Shore A hardness of less than about 60.

In some embodiments, the edible body 12 includes the dental additive. The edible body 12 also includes from about 0.05 mg to about 10.0 mg of astaxanthin. The dental additive is selected from glucose oxidase, L-ascorbic acid phosphate, lactoperooxidase, tetrasodium pyrophosphate, or any combination thereof. The edible body 12 has an average Shore A hardness of less than about 60.

In some embodiments, edible body 12 includes the polishing agent and the dental additive. The edible body 12 also includes from about 0.05 mg to about 10.0 mg of astaxanthin. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The edible body 12 has an average Shore A hardness of less than about 60.

In some embodiments, the edible body 12 includes one or more of the polishing agent, the dental additive, and the hardness reducing ingredient. The edible body 12 also includes from about 0.05 mg to about 10.0 mg of astaxanthin. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The hardness reducing ingredient is chicken meat. The edible body 12 has an average Shore A hardness of less than about 60.

In some embodiments, the edible body 12 includes the polishing agent, the flavor additive, the vegetable oil, the humectant, the dental additive, the food additive, the hardness reducing ingredient, and water. The edible body 12 also includes from about 0.05 mg to about 10.0 mg of astaxanthin. The edible body 12 has an average Shore A hardness of less than about 60.

### A. Multi-Layer Embodiment

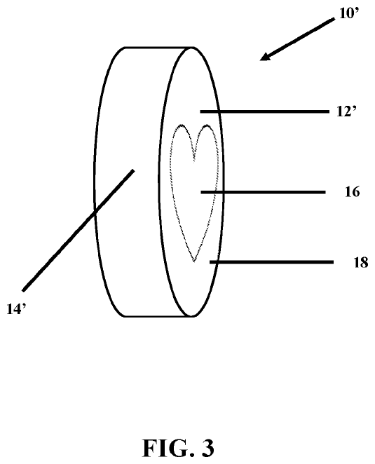

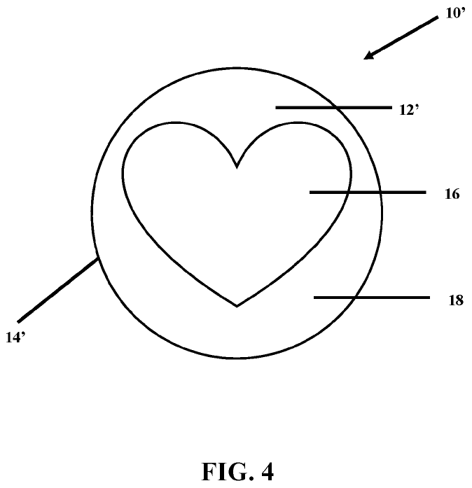

With reference to FIGS. 3 and 4, in some embodiments, the edible body 12′ of the edible soft dental chew 10′ includes a core 16 and an outer layer 18. The outer layer 18 is disposed on the core 16. At least one of the core 16 and the outer layer 18 include the astaxanthin. The outer layer 18 has an average Shore A hardness of less than about 60.

### 1. Core

With continued reference to FIGS. 3 and 4, the core 16 is an edible material. In some embodiments, the core 16 is chewable. The geometrical configuration of a cross-sectional shape defined by the core 16 is not particularly limited, and may be any cross-sectional shape suitable for consumption by a non-human animal. By way of non-limiting example, the cross-sectional shape defined by the core 16 may be a triangle, square, rectangle, oval, circle, square, diamond, heart, star, bone, or any other geometric shape. As shown in FIG. 4, in some embodiments, the cross-sectional shape defined by the core 16 is a heart.

In some embodiments, the core 16 has an average Shore A hardness equal to the average shore A hardness of the outer layer 18. In some embodiments, the core 16 has an average Shore A hardness less than the average shore A hardness of the outer layer 18. In some embodiments, the core 16 has an average Shore A hardness greater than the average shore A hardness of the outer layer 18.

In some embodiments, the core 16 has an average Shore A hardness of less than about 57.5. In some embodiments, the core 16 has an average Shore A hardness of less than about 55. In some embodiments, the core 16 has an average Shore A hardness of less than about 52.5. In some embodiments, the core 16 has an average Shore A hardness of less than about 50. In some embodiments, the core 16 has an average Shore A hardness of less than about 45. In some embodiments, the core 16 has an average Shore A hardness of less than about 40. In some embodiments, the core 16 has an average Shore A hardness of less than about 35. In some embodiments, the core 16 has an average Shore A hardness of less than about 30. In some embodiments, the core 16 has an average Shore A hardness of less than about 25. In some embodiments, the core 16 has an average Shore A hardness of less than about 20. In some embodiments, the core 16 has an average Shore A hardness of less than about 15. In some embodiments, the core 16 has an average Shore A hardness of less than about 10.

In some embodiments, the core 16 has an average Shore 00 hardness of less than about 30. In some embodiments, the core 16 has an average Shore 00 hardness of less than about 20. In some embodiments, the core 16 has an average Shore 00 hardness of less than about 15.

In some embodiments, the core 16 has an average Shore A hardness of from about 10 to about 40. In some embodiments, the core 16 has an average Shore A hardness of from about 12.5 to about 37.5. In some embodiments, the core 16 has an average Shore A hardness of from about 15 to about 35. In some embodiments, the core 16 has an average Shore A hardness of from about 17.5 to about 32.5. In some embodiments, the core 16 has an average Shore A hardness of from about 20 to about 30.

In some embodiments, the core 16 has an average Shore A hardness of from about 10 to about 30. In some embodiments, the core 16 has an average Shore A hardness of from about 10 to about 20. In some embodiments, the core 16 has an average Shore A hardness of from about 20 to about 30. In some embodiments, the core 16 has an average Shore A hardness of from about 20 to about 40. In some embodiments, the core 16 has an average Shore A hardness of from about 30 to about 40. In some embodiments, the core 16 has an average Shore A hardness of from about 30 to about 50. In some embodiments, the core 16 has an average Shore A hardness of from about 40 to about 50.

In some embodiments, the core 16 includes one or more of the polishing agent, the flavor additive, the vegetable oil, the humectant, the dental additive, the food additive, the hardness reducing ingredient, and water.

In some embodiments, the core 16 includes the hardness reducing ingredient as described herein. In some embodiments, the hardness reducing ingredient is chicken meat. For example, the chicken meat may be chicken meal.

In some embodiments, the core 16 comprises from about 1 wt % to about 50 wt % of the hardness reducing ingredient (e.g., chicken), by percent weight of the core. In some embodiments, the core 16 comprises from about 1 wt % to about 50 wt % of the hardness reducing ingredient (e.g., chicken), by percent weight of the core. In some embodiments, the core 16 comprises from about 2 wt % to about 40 wt % of the hardness reducing ingredient (e.g., chicken), by percent weight of the core. In some embodiments, the core 16 comprises from about 4 wt % to about 25 wt % of the hardness reducing ingredient (e.g., chicken), by percent weight of the core. In some embodiments, the core 16 comprises from about 5 wt % to about 20 wt % of the hardness reducing ingredient (e.g., chicken), by percent weight of the core. In some embodiments, the core 16 comprises from about 5 wt % to about 10 wt % of the hardness reducing ingredient (e.g., chicken), by percent weight of the core. In some embodiments, the core 16 comprises from about 5 wt % to about 50 wt % of the hardness reducing ingredient (e.g., chicken), by percent weight of the core. In some embodiments, the core 16 comprises from about 8 wt % to about 20 wt % of the hardness reducing ingredient (e.g., chicken), by percent weight of the core. In some embodiments, the core 16 comprises from about 8 wt % to about 50 wt % of the hardness reducing ingredient (e.g., chicken), by percent weight of the core 16.

In some embodiments, the core 16 includes the polishing agent as described herein. In some embodiments, the core 16 includes a polishing agent selected from sodium bicarbonate, calcium carbonate, dicalcium phosphate, calcium sulfate, silica, iron oxide, hydroxyapatite, aluminum oxide, perlite, plastic particles, or any combination thereof. In some embodiments, the core 16 includes a polishing agent selected from calcium carbonate, dicalcium phosphate, sodium bicarbonate, or any combination thereof. In some embodiments, the polishing agent includes calcium carbonate. In some embodiments, the polishing agent includes dicalcium phosphate. In some embodiments, the polishing agent includes sodium bicarbonate. In some embodiments, the polishing agent includes calcium carbonate, dicalcium phosphate, and sodium bicarbonate. In some embodiments, the polishing agent is a combination of calcium carbonate, dicalcium phosphate, and sodium bicarbonate.

In some embodiments, the core 16 includes the flavor additive as described herein. In some embodiments, the core 16 includes a flavor additive selected from spearmint, peppermint, wintergreen, sassafras, clove, sage, eucalyptus, marjoram, cinnamon, lemon, lime, grapefruit, coconut, orange, vanilla, rosemary, salt, chicken flavor, honey, or any combination thereof. In some embodiments, the core 16 includes a flavor additive selected from clove, cinnamon, vanilla, rosemary, salt, chicken flavor, honey, or any combination thereof. In some embodiments, the flavor additive includes clove. In some embodiments, the flavor additive includes cinnamon. In some embodiments, the flavor additive includes vanilla. In some embodiments, the flavor additive includes rosemary. In some embodiments, the flavor additive includes salt. In some embodiments, the flavor additive includes chicken flavor. In some embodiments, the flavor additive honey. In some embodiments, the flavor additive includes clove, cinnamon, vanilla, rosemary, salt, chicken flavor, and honey. In some embodiments, the flavor additive is a combination of clove, cinnamon, vanilla, rosemary, salt, chicken flavor, and honey.

In some embodiments, the core 16 includes the vegetable oil as described herein. In some embodiments, the core 16 includes a vegetable oil selected from palm oil, soybean oil, canola oil, sunflower seed oil, flaxseed oil, peanut oil, cottonseed oil, palm kernel oil, coconut oil, olive oil, or any combination thereof. In some embodiments, the core 16 includes a vegetable oil selected from selected from canola oil, flaxseed oil, or any combination thereof. In some embodiments, the vegetable includes canola oil. In some embodiments, the vegetable oil includes flaxseed oil. In some embodiments, the vegetable oil includes canola oil and flaxseed oil. In some embodiments, the vegetable oil is a combination of canola oil and flaxseed oil.

In some embodiments, the core 16 includes the humectant as described herein. In some embodiments, the core 16 includes a humectant selected from sucrose, glycerin, glycerol, sorbitol, xylitol, butylene glycol, propylene glycol, or any combination thereof. In some embodiments, the core 16 includes a humectant selected from glycerin, sorbitol, or any combination thereof. In some embodiments, the humectant includes glycerin. In some embodiments, the humectant includes sorbitol. In some embodiments, the humectant includes glycerin and sorbitol. In some embodiments, the humectant is glycerin.

In some embodiments, the core 16 includes the dental additive as described herein. In some embodiments, the core 16 includes a dental additive selected from glucose oxidase, L-ascorbic acid phosphate, lactoperooxidase, tetrasodium pyrophosphate, or any combination thereof. In some embodiments, the dental additive includes glucose oxidase. In some embodiments, the dental additive includes L-ascorbic acid phosphate. In some embodiments, the dental additive includes lactoperooxidase. In some embodiments, the dental additive includes tetrasodium pyrophosphate. In some embodiments, the dental additive includes glucose oxidase, L-ascorbic acid phosphate, and tetrasodium pyrophosphate. In some embodiments, the dental additive is a combination of glucose oxidase, L-ascorbic acid phosphate, and tetrasodium pyrophosphate.

In some embodiments, the core 16 includes the food additive as described herein. In some embodiments, the core 16 includes a food additive selected from cultured dextrose, gelatin, lecithin, rice flour, citric acid, or any combination thereof. In some embodiments, the food additive includes cultured dextrose. In some embodiments, the food additive includes gelatin. In some embodiments, the food additive includes rice flour. In some embodiments, the food additive includes citric acid. In some embodiments, the food additive includes cultured dextrose, gelatin, lecithin, rice flour, and citric acid. In some embodiments, the food additive is a combination of cultured dextrose, gelatin, lecithin, rice flour, and citric acid.

In some embodiments, the core 16 includes water. In some embodiments, the core 16 includes the polishing agent, the flavor additive, the vegetable oil, the humectant, the dental additive, the food additive, the hardness reducing ingredient, and water. In some embodiments, the core 16 includes calcium carbonate, canola oil, cinnamon, citric acid, clove, cultured dextrose, dicalcium phosphate, flaxseed oil, gelatin, glycerin, honey, L-ascorbic acid phosphate, lecithin, chicken meat, rice flour, salt, sodium bicarbonate, tetrasodium pyrophosphate, vanilla, and water.

In some embodiments, the core 16 includes one or more binders and/or fillers as described herein. In some embodiments, the core 16 is an extrudate.

In some embodiments, the core 16 includes astaxanthin in any of the amounts as described herein. In some embodiments, the core 16 is substantially free of astaxanthin.

### 2. Outer Layer

With reference again to FIGS. 3, and 4, the outer layer 18 is an edible material. In some embodiments, the outer layer 18 is chewable. The geometrical configuration of a cross-sectional shape defined by the outer layer 18 is not particularly limited, and may be any cross-sectional shape suitable for consumption by a non-human animal. By way of non-limiting example, the cross-sectional shape defined by the outer layer 18 may be a triangle, square, rectangle, oval, circle, square, diamond, heart, star, bone, or any other shape. As shown in FIG. 4, in some embodiments, the cross-sectional shape defined by the outer layer 18 is a circle.

With continued reference to FIGS. 3 and 4, the outer layer 18 includes an exterior surface 14′ facing away from the core 16. As shown in FIGS. 3 and 4, the exterior surface 14′ may be substantially free of any protrusions configured to remove dental plaque and/or dental calculus from teeth of the non-human animal. In other embodiments, the exterior surface may include protrusions configured to remove dental plaque and/or dental calculus from teeth of the non-human animal, such as the protrusions described herein.

The outer layer 18 is disposed on the core 16. As shown in FIGS. 3 and 4, in some embodiments, the outer layer 18 surrounds the core 16. In other embodiments, the outer layer 18 partially surrounds the core 16. In other words, there may be portions of the core 16 that the outer layer 18 is not disposed on. In some embodiments, one or more intermediate layers are disposed between the core 16 and the outer layer 18. In some embodiments, the outer layer 18 has a red color.

In some embodiments, the outer layer 18 has an average Shore A hardness of less than about 57.5. In some embodiments, the outer layer 18 has an average Shore A hardness of less than about 55. In some embodiments, the outer layer 18 has an average Shore A hardness of less than about 52.5. In some embodiments, the outer layer 18 has an average Shore A hardness of less than about 50. In some embodiments, the outer layer 18 has an average Shore A hardness of less than about 45. In some embodiments, the outer layer 18 has an average Shore A hardness of less than about 40. In some embodiments, the outer layer 18 has an average Shore A hardness of less than about 35. In some embodiments, the outer layer 18 has an average Shore A hardness of less than about 30. In some embodiments, the outer layer 18 has an average Shore A hardness of less than about 25. In some embodiments, the outer layer 18 has an average Shore A hardness of less than about 20. In some embodiments, the outer layer 18 has an average Shore A hardness of less than about 15. In some embodiments, the outer layer 18 has an average Shore A hardness of less than about 10.

In some embodiments, the outer layer 18 has an average Shore 00 hardness of less than about 30. In some embodiments, the outer layer 18 has an average Shore 00 hardness of less than about 20. In some embodiments, the outer layer 18 has an average Shore 00 hardness of less than about 15.

In some embodiments, the outer layer 18 has an average Shore A hardness of from about 40 to about 59. In some embodiments, the outer layer 18 has an average Shore A hardness of from about 42.5 to about 57.5. In some embodiments, the outer layer 18 has an average Shore A hardness of from about 45 to about 55. In some embodiments, the outer layer 18 has an average Shore A hardness of from about 47.5 to about 52.5. In some embodiments, the outer layer 18 has an average Shore A hardness of about 50.

In some embodiments, the outer layer 18 has an average Shore A hardness of from about 10 to about 30. In some embodiments, the outer layer 18 has an average Shore A hardness of from about 10 to about 20. In some embodiments, the outer layer 18 has an average Shore A hardness of from about 20 to about 30. In some embodiments, the outer layer 18 has an average Shore A hardness of from about 20 to about 40. In some embodiments, the outer layer 18 has an average Shore A hardness of from about 30 to about 40. In some embodiments, the outer layer 18 has an average Shore A hardness of from about 30 to about 50. In some embodiments, the outer layer 18 has an average Shore A hardness of from about 40 to about 50.

In some embodiments, the outer layer 18 includes the hardness reducing ingredient as described herein. In some embodiments, the hardness reducing ingredient is chicken meat. For example, the chicken meat may be chicken meal. In other embodiments, the outer layer 18 is substantially free of the hardness reducing ingredient.

In some embodiments, the outer layer 18 comprises from about 1 wt % to about 50 wt % of the hardness reducing ingredient (e.g., chicken), by percent weight of the outer layer. In some embodiments, the outer layer 18 comprises from about 1 wt % to about 50 wt % of the hardness reducing ingredient (e.g., chicken), by percent weight of the outer layer. In some embodiments, the outer layer 18 comprises from about 2 wt % to about 40 wt % of the hardness reducing ingredient (e.g., chicken), by percent weight of the outer layer. In some embodiments, the c outer layer 18 comprises from about 4 wt % to about 25 wt % of the hardness reducing ingredient (e.g., chicken), by percent weight of the outer layer. In some embodiments, the outer layer 18 comprises from about 5 wt % to about 20 wt % of the hardness reducing ingredient (e.g., chicken), by percent weight of the outer layer. In some embodiments, the outer layer 18 comprises from about 5 wt % to about 10 wt % of the hardness reducing ingredient (e.g., chicken), by percent weight of the outer layer. In some embodiments, the outer layer 18 comprises from about 5 wt % to about 50 wt % of the hardness reducing ingredient (e.g., chicken), by percent weight of the outer layer. In some embodiments, the outer layer 18 comprises from about 8 wt % to about 20 wt % of the hardness reducing ingredient (e.g., chicken), by percent weight of the outer layer. In some embodiments, the outer layer 18 comprises from about 8 wt % to about 50 wt % of the hardness reducing ingredient (e.g., chicken), by percent weight of the outer layer.

In some embodiments, the outer layer 18 includes from about 0.01 mg to about 10 mg of astaxanthin. In some embodiments, the outer layer 18 includes from about 0.05 mg to about 10 mg of astaxanthin. In some embodiments, the outer layer 18 includes from about 0.1 mg to about 0.9 mg of astaxanthin. In some embodiments, the outer layer 18 includes from about 0.2 mg to about 0.8 mg of astaxanthin. In some embodiments, the outer layer 18 includes from about 0.25 mg to about 0.75 mg of astaxanthin. In some embodiments, the outer layer 18 includes from about 0.3 mg to about 0.7 mg of astaxanthin. In some embodiments, the outer layer 18 includes from about 0.35 mg to about 0.65 mg of astaxanthin. In some embodiments, the outer layer 18 includes from about 0.4 mg to about 0.6 mg of astaxanthin. In some embodiments, the outer layer 18 includes from about 0.45 mg to about 0.55 mg of astaxanthin. In some embodiments, the outer layer 18 includes about 0.5 mg of astaxanthin. In some embodiments, the outer layer 18 includes from about 0.01 mg to about 4 mg of astaxanthin. In some embodiments, the outer layer 18 includes about 0.2 mg to about 5 mg of astaxanthin. In some embodiments, the outer layer 18 includes from about 0.3 mg to about 4 mg of astaxanthin.

In some embodiments, the outer layer 18 includes from about 0.5 mg to about 2.0 mg of astaxanthin. In some embodiments, the outer layer 18 includes from about 0.6 mg to about 1.9 mg of astaxanthin. In some embodiments, the outer layer 18 includes from about 0.7 mg to about 1.8 mg of astaxanthin. In some embodiments, the outer layer 18 includes from about 0.75 mg to about 1.75 mg of astaxanthin. In some embodiments, the outer layer 18 includes from about 0.8 mg to about 1.7 mg of astaxanthin. In some embodiments, the outer layer 18 includes from about 0.85 mg to about 1.65 mg of astaxanthin. In some embodiments, the outer layer 18 includes from about 0.9 mg to about 1.6 mg of astaxanthin. In some embodiments, the outer layer 18 includes from about 0.95 mg to about 1.55 mg of astaxanthin. In some embodiments, the outer layer 18 includes from about 1.00 mg to about 1.5 mg of astaxanthin. In some embodiments, the outer layer 18 includes from about 1.05 mg to about 1.45 mg of astaxanthin. In some embodiments, the outer layer 18 includes from about 1.10 mg to about 1.40 mg of astaxanthin. In some embodiments, the outer layer 18 includes from about 1.15 mg to about 1.35 mg of astaxanthin. In some embodiments, the outer layer 18 includes from about 1.20 mg to about 1.30 mg of astaxanthin. In some embodiments, the outer layer 18 includes about 1.25 mg of astaxanthin.

In some embodiments, the outer layer 18 includes from about 0.5 mg to about 1.5 mg of astaxanthin. In some embodiments, the outer layer 18 includes about 1.0 mg of astaxanthin. In some embodiments, the outer layer 18 includes from about 1.5 mg to about 2.5 mg of astaxanthin. In some embodiments, the outer layer 18 includes about 2.0 mg of astaxanthin. In some embodiments, the outer layer 18 includes from about 2.0 mg to about 3.0 mg of astaxanthin. In some embodiments, the outer layer 18 includes about 2.5 mg of astaxanthin. In some embodiments, the outer layer 18 includes from about 4.5 mg to about 5.5 mg of astaxanthin. In some embodiments, the outer layer 18 includes about 5.0 mg of astaxanthin.

In some embodiments, the outer layer 18 includes one or more binders and/or fillers. When present, the binders and/or fillers may be any binders and/or fillers suitable for consumption by a non-human animal. In some embodiments, the outer layer 18 is an extrudate. In some embodiments, each of the core 16 and the outer layer 18 independently include one or more binders and/or fillers.

In some embodiments, the outer layer 18 includes one or more of the polishing agent, the flavor additive, the vegetable oil, the humectant, the dental additive, the food additive, the hardness reducing ingredient, and water as described herein. In other embodiments, the outer layer 18 is substantially free of any of the polishing agent, the flavor additive, the vegetable oil, the humectant, the dental additive, the food additive, and the hardness reducing ingredient as described herein.

In some embodiments, the edible soft dental chew 10′ includes the core 16 and the outer layer 18. The core 16 includes one or more of the polishing agent, the flavor additive, the vegetable oil, the humectant, the dental additive, the food additive, the hardness reducing ingredient, and water. The outer layer 18 includes from about 0.05 mg to about 10.0 mg of astaxanthin. The outer layer 18 has an average Shore A hardness of less than about 60. The core 16 has an average Shore A hardness of less than about 40.

In some embodiments, the edible soft dental chew 10′ includes the core 16 and the outer layer 18. The core 16 includes the hardness reducing ingredient. The outer layer 18 includes from about 0.05 mg to about 10.0 mg of astaxanthin. The outer layer 18 has an average Shore A hardness of less than about 60. The core 16 has an average Shore A hardness of less than about 40.

In some embodiments, the edible soft dental chew 10′ includes the core 16 and the outer layer 18. The core 16 includes the polishing agent. The outer layer 18 includes from about 0.05 mg to about 10.0 mg of astaxanthin. The polishing agent is selected from calcium carbonate, dicalcium phosphate, sodium bicarbonate, or any combination thereof. The outer layer 18 has an average Shore A hardness of less than about 60. The core 16 has an average Shore A hardness of less than about 40.

In some embodiments, the edible soft dental chew 10′ includes the core 16 and the outer layer 18. The core 16 includes the dental additive. The outer layer 18 includes from about 0.05 mg to about 10.0 mg of astaxanthin. The dental additive is selected from glucose oxidase, L-ascorbic acid phosphate, lactoperooxidase, tetrasodium pyrophosphate, or any combination thereof. The outer layer 18 has an average Shore A hardness of less than about 60. The core 16 has an average Shore A hardness of less than about 40.

In some embodiments, the edible soft dental chew 10′ includes the core 16 and the outer layer 18. The core 16 includes the polishing agent and the dental additive. The outer layer 18 includes from about 0.05 mg to about 10.0 mg of astaxanthin. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The outer layer 18 has an average Shore A hardness of less than about 60. The core 16 has an average Shore A hardness of less than about 40.

In some embodiments, the edible soft dental chew 10′ includes the core 16 and the outer layer 18. The core 16 includes one or more of the polishing agent, the dental additive, and the hardness reducing ingredient. The outer layer 18 includes from about 0.05 mg to about 10.0 mg of astaxanthin. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The hardness reducing ingredient is chicken meat. The outer layer 18 has an average Shore A hardness of less than about 60. The core 16 has an average Shore A hardness of less than about 40.

In some embodiments, the edible soft dental chew 10′ includes the core 16 and the outer layer 18. The core 16 includes the polishing agent, the flavor additive, the vegetable oil, the humectant, the dental additive, the food additive, the hardness reducing ingredient, and water. The outer layer 18 includes from about 0.05 mg to about 10.0 mg of astaxanthin. The outer layer 18 has an average Shore A hardness of less than about 60. The core 16 has an average Shore A hardness of less than about 40.

### 3. Additional Embodiments

In some embodiments, the edible soft dental chew 10′ includes one or more intermediate layers disposed between the core 16 and the outer layer 18. When present, the one or more intermediate layers at least partially surround the core 16. In some embodiments, the one or more intermediate layers entirely surround the core 16. In some embodiments, the one or more intermediate layers include one or more of astaxanthin, the polishing agent, the flavor additive, the vegetable oil, the humectant, the dental additive, the food additive, the hardness reducing ingredient, and water as described herein. In other embodiments, the one or more intermediate layers are substantially free of any of astaxanthin, the polishing agent, the flavor additive, the vegetable oil, the humectant, the dental additive, the food additive, and the hardness reducing ingredient as described herein. The one or more intermediate layers may have an average Shore A hardness that is equal to or less than the average Shore A hardness of the outer layer 18.

Another aspect of the present invention provides an edible soft dental chew that includes a core and an outer layer disposed on the core. At least one of the core and the outer layer includes astaxanthin. The outer layer has an average Shore A hardness of less than about 60. At least one of the core and the outer layer includes one or more of the polishing agent, the flavor additive, the vegetable oil, the humectant, the dental additive, the food additive, the hardness reducing ingredient, and water as described herein. In some embodiments, the outer layer includes astaxanthin and one or more of the polishing agent, the flavor additive, the vegetable oil, the humectant, the dental additive, the food additive, the hardness reducing ingredient, and water. In some embodiments, the core includes astaxanthin and one or more of the polishing agent, the flavor additive, the vegetable oil, the humectant, the dental additive, the food additive, the hardness reducing ingredient, and water. In other embodiments, the core includes astaxanthin and the outer layer includes one or more of the polishing agent, the flavor additive, the vegetable oil, the humectant, the dental additive, the food additive, the hardness reducing ingredient, and water.

### III. Methods of Preventing or Inhibiting Dental Plaque Accumulation and/or Dental Calculus Formation

Another aspect of the present inventions provides a method preventing or inhibiting dental plaque accumulation and/or dental calculus formation in a subject in need thereof. The method includes administering to the subject at least one edible soft dental chew at least once per day. The edible soft dental chew may be any edible soft dental chew as described herein. The subject is a non-human animal. The edible soft dental chew has an average Shore A hardness of less than about 60.

In some implementations, the edible soft dental chew includes an edible body that includes astaxanthin as described herein.

In some implementations, the edible body includes a core and an outer layer disposed on the core as described herein. In some implementations, the outer layer has an average Shore A hardness of less than about 60. In some implementations, the core has an average shore A hardness of less than about 40. In some implementations, the core and/or the outer layer may have any average Shore A hardness as described herein.

In some implementations, the core includes the hardness reducing ingredient. In some implementations, the outer layer includes the hardness reducing ingredient. In some implementations, the hardness reducing ingredient may be chicken meat.

In some implementations, the non-human animal has a weight of up to about 15.0 lbs. In some implementations, the non-human animal has a weight of from about 15.0 lbs to about 30.0 lbs. In some implementations, the non-human animal has a weight of from about 30.0 lbs to about 50.0 lbs. In some implementations, the non-human animal has a weight of greater than about 50.0 lbs. For example, the non-human animal may have a weight of from about 50.0 lbs to about 350 lbs. In some implementations, the non-human animal has a weight of up to about 8.0 lbs. In some implementations, the non-human animal has a weight of from about 8.0 lbs to about 20.0 lbs. In some implementations, the non-human animal has a weight of from 20.0 lbs to about 40.0 lbs. In some implementations, the non-human animal has a weight of greater than about 40.0 lbs.

In some implementations, the edible body includes from about 0.01 mg to about 10.0 mg of astaxanthin. In other implementations, the edible body includes any amount of astaxanthin as described herein.

In some implementations, the outer layer of the edible body includes from about 0.01 mg to about 10.0 mg of astaxanthin. In other implementations, the outer layer of the edible body includes any amount of astaxanthin as described herein.

In some implementations, the edible soft dental chew is administered at a dose based on the weight of the non-human animal and the amount of astaxanthin in the edible soft dental chew.

In some implementations, the non-human animal has a weight of up to 350 lbs, the edible body includes from about 0.05 mg to about 10.0 mg of astaxanthin, and the subject is administered at least one edible soft dental chew at least once per day. In some implementations, the subject is administered one edible soft dental chew once per day. In some implementations, the subject is administered two edible soft dental chews once per day. In some implementations, the subject is administered one edible soft dental chew twice per day. In some implementations, the subject is administered two edible soft dental chews twice per day.

In some implementations, the non-human animal has a weight of up to 350 lbs, the outer layer of the edible body includes from about 0.05 mg to about 10.0 mg of astaxanthin, and the subject is administered at least one edible soft dental chew at least once per day. In some implementations, the subject is administered one edible soft dental chew once per day. In some implementations, the subject is administered two edible soft dental chews once per day. In some implementations, the subject is administered one edible soft dental chew twice per day. In some implementations, the subject is administered two edible soft dental chews twice per day.

In some implementations, the non-human animal has a weight of up to about 15.0 lbs, the edible body includes from about 0.2 mg to about 0.8 mg of astaxanthin, and the subject is administered at least one edible soft dental chew at least once per day. In some implementations, the edible body includes from about 0.3 mg to about 0.7 mg of astaxanthin. In some implementations, the edible body includes from about 0.4 mg to about 0.6 mg of astaxanthin. In some implementations, the edible body includes about 0.5 mg of astaxanthin. In some implementations, the subject is administered one edible soft dental chew twice per day.

In some implementations, the non-human animal has a weight of up to about 15.0 lbs, the outer layer of the edible body includes from about 0.2 mg to about 0.8 mg of astaxanthin, and the subject is administered at least one edible soft dental chew at least once per day. In some implementations, the outer layer of the edible body includes from about 0.3 mg to about 0.7 mg of astaxanthin. In some implementations, the outer layer of the edible body includes from about 0.4 mg to about 0.6 mg of astaxanthin. In some implementations, the outer layer of the edible body includes about 0.5 mg of astaxanthin. In some implementations, the subject is administered one edible soft dental chew twice per day.

In some implementations, the non-human animal has a weight of from about 15.0 lbs to about 30.0 lbs, the edible body includes from about 0.2 mg to about 0.8 mg of astaxanthin, and the subject is administered at least one edible soft dental chew at least once per day. In some implementations, the edible body includes from about 0.3 mg to about 0.7 mg of astaxanthin. In some implementations, the edible body includes from about 0.4 mg to about 0.6 mg of astaxanthin. In some implementations, the edible body includes about 0.5 mg of astaxanthin. In some implementations, the subject is administered two edible soft dental chews twice per day.

In some implementations, the non-human animal has a weight of from about 15.0 lbs to about 30.0 lbs, the outer layer of the edible body includes from about 0.2 mg to about 0.8 mg of astaxanthin, and the subject is administered at least one edible soft dental chew at least once per day. In some implementations, the outer layer of the edible body includes from about 0.3 mg to about 0.7 mg of astaxanthin. In some implementations, the outer layer of the edible body includes from about 0.4 mg to about 0.6 mg of astaxanthin. In some implementations, the outer layer of the edible body includes about 0.5 mg of astaxanthin. In some implementations, the subject is administered two edible soft dental chews twice per day.

In some implementations, the non-human animal has a weight of from about 30.0 lbs to about 50.0 lbs, the edible body includes from about 0.75 mg to about 1.75 mg of astaxanthin, and the subject is administered at least one edible soft dental chew at least once per day. In some implementations, the edible body includes from about 1.0 mg to about 1.5 mg of astaxanthin. In some implementations, the edible body includes from about 1.2 mg to about 1.3 mg of astaxanthin. In some implementations, the edible body includes about 1.25 mg of astaxanthin. In some implementations, the subject is administered one edible soft dental chew twice per day.

In some implementations, the non-human animal has a weight of from about 30.0 lbs to about 50.0 lbs, the outer layer of the edible body includes from about 0.75 mg to about 1.75 mg of astaxanthin, and the subject is administered at least one edible soft dental chew at least once per day. In some implementations, the outer layer of the edible body includes from about 1.0 mg to about 1.5 mg of astaxanthin. In some implementations, the outer layer of the edible body includes from about 1.2 mg to about 1.3 mg of astaxanthin. In some implementations, the outer layer of the edible body includes about 1.25 mg of astaxanthin. In some implementations, the subject is administered one edible soft dental chew twice per day.

In some implementations, the non-human animal has a weight of from about 30.0 lbs to about 50.0 lbs, the edible body includes from about 0.75 mg to about 1.75 mg of astaxanthin, and the subject is administered at least one edible soft dental chew at least once per day. In some implementations, the edible body includes from about 1.0 mg to about 1.5 mg of astaxanthin. In some implementations, the edible body includes from about 1.2 mg to about 1.3 mg of astaxanthin. In some implementations, the edible body includes about 1.25 mg of astaxanthin. In some implementations, the subject is administered one edible soft dental chew twice per day.

In some implementations, the non-human animal has a weight of from about 30.0 lbs to about 50.0 lbs, the outer layer of the edible body includes from about 0.75 mg to about 1.75 mg of astaxanthin, and the subject is administered at least one edible soft dental chew at least once per day. In some implementations, the outer layer of the edible body includes from about 1.0 mg to about 1.5 mg of astaxanthin. In some implementations, the outer layer of the edible body includes from about 1.2 mg to about 1.3 mg of astaxanthin. In some implementations, the outer layer of the edible body includes about 1.25 mg of astaxanthin. In some implementations, the subject is administered two edible soft dental chews twice per day.

In some embodiments, the non-human animal has a weight of up to about 8 lbs, wherein the outer layer comprises from about 0.2 mg to about 0.8 mg of astaxanthin, and wherein the subject is administered at least one edible soft dental chew at least once per day. In some embodiments, the subject is administered one edible soft dental chew twice per day.

In some embodiments, the non-human animal has a weight of from about 8 lbs to about 20 lbs, wherein the outer layer comprises from about 1.8 mg to about 2 mg of astaxanthin, and wherein the subject is administered at least one edible soft dental chew at least once per day. In some embodiments, the subject is administered two edible soft dental chews twice per day.

In some embodiments, the non-human animal has a weight of from about 20 lbs to about 40 lbs, wherein the outer layer comprises from about 2.6 mg to about 4 mg of astaxanthin, and wherein the subject is administered at least one edible soft dental chew at least once per day. In some embodiments, the subject is administered one edible soft dental chew twice per day.

In some embodiments, the non-human animal has a weight of greater than about 40 lbs, wherein the outer layer comprises about 4 mg of astaxanthin, and wherein the subject is administered at least one edible soft dental chew at least once per day. In some embodiments, the subject is administered two edible soft dental chews twice per day.

In some implementations, the non-human animal is at least 12 weeks old. In other implementations, the non-human animal is from about 0 to about 12 weeks old.

In some implementations, the non-human animal has one or more of missing teeth, deciduous teeth, and cracked teeth. For example, the non-human animal has missing teeth. In other implementations, the non-human animal has deciduous teeth. In some implementations, the non-human animal has cracked teeth. In some implementations, the non-human animal has a preference for soft food/chews.

In some implementations, the non-human animal is a canine (e.g., a dog) or a feline (e.g., a cat). In some implementations, the non-human animal is a dog. In some implementations, teeth of the non-human animal are cleaned by a veterinarian prior to the administration of the at least one edible soft dental chew. In some implementations, teeth of the non-human animal are substantially free of dental plaque prior to the administration of the at least one edible soft dental chew. In some implementations, teeth of the non-human animal are substantially free of dental calculus prior to the administration of the at least one edible soft dental chew. In other implementations, teeth of the non-human animal are not cleaned prior to the administration of the at least one edible soft dental chew.

In some implementations, the subject (i.e., the non-human animal) experiences an increase in salivary immunoglobulin A (sIgA) concentration following onset of administration. In some implementations, the subject experiences an increase in sigA concentration of at least about 1% following onset of administration. In some implementations, the subject experiences an increase in sIgA concentration of at least about 2.5% following onset of administration. In some implementations, the subject experiences an increase in sIgA concentration of at least about 5% following onset of administration. In some implementations, the subject experiences an increase in sIgA concentration of at least about 10% following onset of administration. In some implementations, the subject experiences an increase in sIgA concentration of at least about 0.1%, 0.5%, 1%, 1.5%, 2%, 2.5%, 3%, 3.5%, 4%, 4.5%, 5%, 5.5%, 6%, 6.5%, 7%, 7.5%, 8% 8.5%, 9%, 9.5%, 10%, 11%, 12%, 13%, 14%, 15%, 16%, 17%, 18%, 19%, 20%, 25%, 30%, 35%, 40%, 45%, 50%, 60%, 70%, 80%, 90%, or 99% following onset of administration.

In some implementations, the subject experiences an increase in sIgA concentration about 1 day following onset of administration. In some implementations, the subject experiences an increase in sIgA concentration about 2 days following onset of administration. In some implementations, the subject experiences an increase in sIgA concentration about 5 days following onset of administration. In some implementations, the subject experiences an increase in sIgA concentration about 10 days following onset of administration. In some implementations, the subject experiences an increase in sIgA concentration about 20 days following onset of administration. In some implementations, the subject experiences an increase in sIgA concentration about 30 days following onset of administration. In some implementations, the subject experiences an increase in sIgA concentration about 50 days following onset of administration. In some implementations, the subject experiences an increase in sIgA concentration about 60 days following onset of administration. In some implementations, the subject experiences an increase in sIgA concentration about 90 days following onset of administration.

In some implementations, the subject does not experience a reduction in sIgA concentration when exposed to increased levels of stress (e.g., physical and/or mental stress) following onset of administration. In some implementations, the subject experiences a reduced reduction in sIgA concentration when exposed to increased levels of stress following onset of administration as compared to a subject that is not administered at least one edible soft dental chew at least once per day.

In some implementations, the subject experiences reduced dental plaque accumulation as compared to a subject that is not administered at least one edible soft dental chew at least once per day. In some implementations, the subject experiences reduced dental calculus formation as compared to a subject that is not administered at least one edible soft dental chew at least once per day.

In some implementations, at least one edible soft dental chew is administered at least once per day for about 2 days. In some implementations, at least one edible soft dental chew is administered at least once per day for about 5 days. In some implementations, at least one edible soft dental chew is administered at least once per day for about 10 days. In some implementations, at least one edible soft dental chew is administered at least once per day for about 20 days. In some implementations, at least one edible soft dental chew is administered at least once per day for about 30 days. In some implementations, at least one edible soft dental chew is administered at least once per day for about 50 days. In some implementations, at least one edible soft dental chew is administered at least once per day for about 60 days. In some implementations, at least one edible soft dental chew is administered at least once per day for about 90 days. In some implementations, at least one edible soft dental chew is administered at least once per day for about 1 year. In some implementations, at least one edible soft dental chew is administered twice per day for about 2 days. In some implementations, at least one edible soft dental chew is administered twice per day for about 5 days. In some implementations, at least one edible soft dental chew is administered twice per day for about 10 days. In some implementations, at least one edible soft dental chew is administered twice per day for about 20 days. In some implementations, at least one edible soft dental chew is administered twice per day for about 30 days. In some implementations, at least one edible soft dental chew is administered twice per day for about 50 days. In some implementations, at least one edible soft dental chew is administered twice per day for about 60 days. In some implementations, at least one edible soft dental chew is administered twice per day for about 90 days. In some implementations, at least one edible soft dental chew is administered twice per day for about 1 year.

In some implementations, the method of preventing or inhibiting dental plaque accumulation and/or dental calculus formation includes administering to the subject at least one edible soft dental chew 10 at least once per day. The subject is a canine or a feline. The edible soft dental chew 10 includes the edible body 12 that includes astaxanthin. The edible body 12 includes one or more of the polishing agent, the flavor additive, the vegetable oil, the humectant, the dental additive, the food additive, the hardness reducing ingredient, and water. The edible body 12 includes from about 0.05 mg to about 10.0 mg of the astaxanthin. The edible body 12 has an average Shore A hardness of less than about 60.

In some implementations, the method of preventing or inhibiting dental plaque accumulation and/or dental calculus formation includes administering to the subject at least one edible soft dental chew 10′ at least once per day. The subject is a canine or a feline. The edible soft dental chew 10′ includes the edible body 12′ that includes astaxanthin. The edible body 12′ includes the core 16 and outer layer 18 disposed on the core 16. The core 16 includes one or more of the polishing agent, the flavor additive, the vegetable oil, the humectant, the dental additive, the food additive, the hardness reducing ingredient, and water. The outer layer 18 includes from about 0.05 mg to about 10.0 mg of the astaxanthin. The outer layer 18 has an average Shore A hardness of less than about 60. The core 16 has an average Shore A hardness of less than about 40.

In some implementations, the method of preventing or inhibiting dental plaque accumulation and/or dental calculus formation includes administering to the subject at least one edible soft dental chew 10 at least once per day. The subject is a canine or a feline. The edible soft dental chew 10 includes the edible body 12 that includes astaxanthin. The edible body 12 includes the hardness reducing ingredient. The edible body 12 includes from about 0.05 mg to about 10.0 mg of the astaxanthin. The edible body 12 has an average Shore A hardness of less than about 60.

In some implementations, the method of preventing or inhibiting dental plaque accumulation and/or dental calculus formation includes administering to the subject at least one edible soft dental chew 10′ at least once per day. The subject is a canine or a feline. The edible soft dental chew 10′ includes the edible body 12′ that includes astaxanthin. The edible body 12′ includes the core 16 and outer layer 18 disposed on the core 16. The core 16 includes the hardness reducing ingredient. The outer layer 18 includes from about 0.05 mg to about 10.0 mg of the astaxanthin. The outer layer 18 has an average Shore A hardness of less than about 60. The core 16 has an average Shore A hardness of less than about 40.

In some implementations, the method of preventing or inhibiting dental plaque accumulation and/or dental calculus formation includes administering to the subject at least one edible soft dental chew 10 at least once per day. The subject is a canine or a feline. The edible soft dental chew 10 includes the edible body 12 that includes astaxanthin. The edible body 12 includes the polishing agent. The polishing agent is selected from calcium carbonate, dicalcium phosphate, sodium bicarbonate, or any combination thereof. The edible body 12 includes from about 0.05 mg to about 10.0 mg of the astaxanthin. The edible body 12 has an average Shore A hardness of less than about 60.

In some implementations, the method of preventing or inhibiting dental plaque accumulation and/or dental calculus formation includes administering to the subject at least one edible soft dental chew 10′ at least once per day. The subject is a canine or a feline. The edible soft dental chew 10′ includes the edible body 12′ that includes astaxanthin. The edible body 12′ includes the core 16 and outer layer 18 disposed on the core 16. The core 16 includes the polishing agent. The polishing agent is selected from calcium carbonate, dicalcium phosphate, sodium bicarbonate, or any combination thereof. The outer layer 18 includes from about 0.05 mg to about 10.0 mg of the astaxanthin. The outer layer 18 has an average Shore A hardness of less than about 60. The core 16 has an average Shore A hardness of less than about 40.

In some implementations, the method of preventing or inhibiting dental plaque accumulation and/or dental calculus formation includes administering to the subject at least one edible soft dental chew 10 at least once per day. The subject is a canine or a feline. The edible soft dental chew 10 includes the edible body 12 that includes astaxanthin. The edible body 12 includes the dental additive. The dental additive is selected from glucose oxidase, L-ascorbic acid phosphate, lactoperooxidase, tetrasodium pyrophosphate, or any combination thereof. The edible body 12 includes from about 0.05 mg to about 10.0 mg of the astaxanthin. The edible body 12 has an average Shore A hardness of less than about 60.

In some implementations, the method of preventing or inhibiting dental plaque accumulation and/or dental calculus formation includes administering to the subject at least one edible soft dental chew 10′ at least once per day. The subject is a canine or a feline. The edible soft dental chew 10′ includes the edible body 12′ that includes astaxanthin. The edible body 12′ includes the core 16 and outer layer 18 disposed on the core 16. The core 16 includes the dental additive. The dental additive is selected from glucose oxidase, L-ascorbic acid phosphate, lactoperooxidase, tetrasodium pyrophosphate, or any combination thereof. The outer layer 18 includes from about 0.05 mg to about 10.0 mg of the astaxanthin. The outer layer 18 has an average Shore A hardness of less than about 60. The core 16 has an average Shore A hardness of less than about 40.

In some implementations, the method of preventing or inhibiting dental plaque accumulation and/or dental calculus formation includes administering to the subject at least one edible soft dental chew 10 at least once per day. The subject is a canine or a feline. The edible soft dental chew 10 includes the edible body 12 that includes astaxanthin. The edible body 12 includes the polishing agent and the dental additive. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The edible body 12 includes from about 0.05 mg to about 10.0 mg of the astaxanthin. The edible body 12 has an average Shore A hardness of less than about 60.

In some implementations, the method of preventing or inhibiting dental plaque accumulation and/or dental calculus formation includes administering to the subject at least one edible soft dental chew 10′ at least once per day. The subject is a canine or a feline. The edible soft dental chew 10′ includes the edible body 12′ that includes astaxanthin. The edible body 12′ includes the core 16 and outer layer 18 disposed on the core 16. The core 16 includes the polishing agent and the dental additive. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The outer layer 18 includes from about 0.05 mg to about 10.0 mg of the astaxanthin. The outer layer 18 has an average Shore A hardness of less than about 60. The core 16 has an average Shore A hardness of less than about 40.

In some implementations, the method of preventing or inhibiting dental plaque accumulation and/or dental calculus formation includes administering to the subject at least one edible soft dental chew 10 at least once per day. The subject is a canine or a feline. The edible soft dental chew 10 includes the edible body 12 that includes astaxanthin. The edible body 12 includes the polishing agent, the dental additive, and the hardness reducing ingredient. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The hardness reducing ingredient is chicken meat. The edible body 12 includes from about 0.05 mg to about 10.0 mg of the astaxanthin. The edible body 12 has an average Shore A hardness of less than about 60.

In some implementations, the method of preventing or inhibiting dental plaque accumulation and/or dental calculus formation includes administering to the subject at least one edible soft dental chew 10′ at least once per day. The subject is a canine or a feline. The edible soft dental chew 10′ includes the edible body 12′ that includes astaxanthin. The edible body 12′ includes the core 16 and outer layer 18 disposed on the core 16. The core 16 includes the polishing agent, the dental additive, and the hardness reducing ingredient. The polishing agent is selected from calcium carbonate, dicalcium phosphate, sodium bicarbonate, or any combination thereof. The dental additive is selected from glucose oxidase, L-ascorbic acid phosphate, lactoperooxidase, tetrasodium pyrophosphate, or any combination thereof. The hardness reducing ingredient is chicken meat. The outer layer 18 includes from about 0.05 mg to about 10.0 mg of the astaxanthin. The outer layer 18 has an average Shore A hardness of less than about 60. The core 16 has an average Shore A hardness of less than about 40.

In some implementations, the method of preventing or inhibiting dental plaque accumulation and/or dental calculus formation includes administering to the subject at least one edible soft dental chew 10 at least once per day. The subject is a canine or a feline. The edible soft dental chew 10 includes the edible body 12 that includes astaxanthin. The edible body 12 includes the polishing agent, the flavor additive, the vegetable oil, the humectant, the dental additive, the food additive, the hardness reducing ingredient, and water. The edible body 12 includes from about 0.05 mg to about 10.0 mg of the astaxanthin. The edible body 12 has an average Shore A hardness of less than about 60.

In some implementations, the method of preventing or inhibiting dental plaque accumulation and/or dental calculus formation includes administering to the subject at least one edible soft dental chew 10′ at least once per day. The subject is a canine or a feline. The edible soft dental chew 10′ includes the edible body 12′ that includes astaxanthin. The edible body 12′ includes the core 16 and outer layer 18 disposed on the core 16. The core 16 includes the polishing agent, the flavor additive, the vegetable oil, the humectant, the dental additive, the food additive, the hardness reducing ingredient, and water. The outer layer 18 includes from about 0.05 mg to about 10.0 mg of the astaxanthin. The outer layer 18 has an average Shore A hardness of less than about 60. The core 16 has an average Shore A hardness of less than about 40.

Another aspect of the invention provides a method of preventing or inhibiting dental plaque accumulation in a subject in need thereof. The method includes administering the edible soft dental chew as described herein. The subject is as described herein.

Another aspect of the invention provides a method of preventing or inhibiting dental calculus formation in a subject in need thereof. The method includes administering the edible soft dental chew as described herein. The subject is as described herein.

Another aspect of the invention provides a method of preventing or inhibiting dental plaque accumulation and dental calculus formation in a subject in need thereof. The method includes administering the edible soft dental chew as described herein. The subject is as described herein.

### IV. Process for Preparing an Edible Soft Dental Chew

Another aspect of the present invention provides a process for preparing an edible soft dental chew 10 as described herein. The edible soft dental chew 10 includes the edible body 12 that includes astaxanthin. The edible body 12 has an average Shore A hardness of less than about 60. The process includes extruding the edible body 12 to form the edible soft dental chew 10.

In some implementations, the step of extruding the edible body 12 is performed by a single screw extruder. In other implementations, the step of extruding the edible body 12 is performed by a twin-screw extruder.

In some implementations, the step of extruding the edible body 12 further includes cooking an edible body composition and extruding the edible body composition to form the edible soft dental chew 10. In some implementations, the steps of cooking the edible body composition and extruding the edible body composition are performed simultaneously. In other implementations, the step of extruding the edible body composition is performed after the step of cooking the edible body composition. In some implementations, the steps of cooking the edible body composition and extruding the edible body composition are performed in a single extruder. In other implementations, the step of cooking the edible body composition is performed in a first extruder and the step of extruding the edible body composition is performed in a second extruder.

Another aspect of the present invention provides a process for preparing an edible soft dental chew 10′ as described herein. The edible soft dental chew 10′ includes the edible body 12′ that includes astaxanthin. The edible body 12′ includes the core 16 and the outer layer 18 disposed on the core 16. At least one of the core 16 and the outer layer 18 includes the astaxanthin. The outer layer 18 has an average Shore A hardness value of less than about 60. The process includes extruding the core 16. The process also includes extruding the outer layer 18 on the core 16 the form the edible soft dental chew 10′. In some implementations, the core 16 has an average Shore A hardness of less than about 40.

In some implementations, the step of extruding the core 16 and the step of extruding the outer layer 18 are performed simultaneously. In other implementations, the step of extruding the outer layer 18 is performed after the step of extruding the core 16.

In some implementations, the step of extruding the core 16 and the step of extruding the outer layer 18 are performed by a single screw extruder. In other implementations, the step of extruding the core 16 and the step of extruding the outer layer 18 are preformed by a twin-screw extruder.

In some implementations, the step of extruding the core 16 further includes cooking a core composition and extruding the core composition to form the core 16. In some implementations, the steps of cooking the core composition and extruding the core composition are performed simultaneously. In other implementations, the step of extruding the core composition is performed after the step of cooking the core composition. In some implementations, the steps of cooking the core composition and extruding the core composition are performed in a single extruder. In other implementations, the step of cooking the core composition is performed in a first extruder and the step of extruding the core composition is performed in a second extruder.

In some implementations, the step of extruding the outer layer 18 further includes cooking an outer layer composition and extruding the outer layer composition to form the outer layer 18. In some implementations, the steps of cooking the outer layer composition and extruding the outer layer composition are performed simultaneously. In other implementations, the step of extruding the outer layer composition is performed after the step of cooking the outer layer composition. In some implementations, the steps of cooking the outer layer composition and extruding the outer layer composition are performed in a single extruder. In other implementations, the step of cooking the outer layer composition is performed in a first extruder and the step of extruding the outer layer composition is performed in a second extruder.

### V. Examples

In order that the invention described herein may be more fully understood, the following examples are set forth. The examples described in this application are offered to illustrate the edible soft dental chews and methods provided herein and are not to be construed in any way as limiting their scope.

### Example 1: Exemplary Edible Soft Dental Chews

Exemplary edible soft dental chews are set forth in Table 1 below and were prepared according to processes described herein. Average Shore A hardness for each exemplary edible soft dental chew was determined with a Shore A durometer.

TABLE 1
Exemplary edible soft dental chews

| | | | Intended |
|---|---|---|---|
| | | Average | non-human |
| Example | | Shore A | animal |
| no. | Components Include | Hardness | weight |
| 1 | Astaxanthin (0.5 mg), calcium carbonate, | ~50 | Up to 15.0 lbs |
| | canola oil, cinnamon, citric acid, clove, | | and 15.0 lbs |
| | cultured dextrose, dicalcium phosphate, | | to about |
| | flaxseed oil, gelatin, glycerin, honey, | | 30.0 lbs |
| | L-ascorbic acid phosphate, lecithin, rice flour, | | |
| | salt, sodium bicarbonate, tetrasodium | | |
| | pyrophosphate, vanilla, chicken meal, and | | |
| | water | | |
| 2 | Astaxanthin (1.25 mg), calcium carbonate, | ~50 | 30.0 lbs to |
| | canola oil, cinnamon, citric acid, clove, | | 50.0 lbs and |
| | cultured dextrose, dicalcium phosphate, | | greater than |
| | flaxseed oil, gelatin, glycerin, honey, | | 50.0 lbs |
| | L-ascorbic acid phosphate, lecithin, rice flour, | | |
| | salt, sodium bicarbonate, tetrasodium | | |
| | pyrophosphate, vanilla, chicken meal, and | | |
| | water | | |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

All issued patents, published patent applications, journal articles, and other publications, which are referred to herein, are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. An edible soft dental chew for preventing or inhibiting dental plaque accumulation and/or dental calculus formation in a non-human animal, the edible soft dental chew comprising:
an edible body comprising astaxanthin,
wherein the edible body has an average Shore A hardness of less than about 60.

2. The edible soft dental chew of claim 1, wherein the edible body comprises from about 0.01 mg to about 4 mg of the astaxanthin.

3. The edible soft dental chew of claim 1 or 2, wherein the edible body comprises a hardness reducing ingredient.

4. The edible soft dental chew of claim 3, wherein the hardness reducing ingredient is meat.

5. The edible soft chew of claim 4, wherein the meat is chicken meat.

6. The edible soft chew of claim 5, wherein the chicken meat is chicken meal.

7. The edible soft chew of claim 5 or 6, wherein the chicken meat is present in an amount of from about 5 wt % to about 10 wt %, by percent of the weight of the edible soft chew.

8. The edible soft chew of any one of claims 1-7, wherein the edible body has an average Shore A hardness of less than about 50.

9. The edible soft dental chew of claim 1, wherein the edible body further comprises:
a core; and
an outer layer disposed on the core,
wherein at least one of the core and the outer layer comprises the astaxanthin, and wherein the outer layer has an average Shore A hardness of less than about 50.

10. The edible soft dental chew of claim 9, wherein the core further comprises one or more of a polishing agent, a flavor additive, a vegetable oil, a humectant, a dental additive, a food additive, a hardness reducing ingredient, and water.

11. The edible soft dental chew of claim 9 or 10, wherein the core has an average Shore A hardness of less than about 50.

12. The edible soft dental chew of any one of claims 9-11, wherein the outer layer comprises the astaxanthin.

13. The edible soft dental chew of any one of claims 9-12, wherein the outer layer comprises from about 0.01 mg to about 10 mg of astaxanthin.

14. The edible soft dental chew of any one of claims 9-13, wherein the outer layer comprises from about 0.2 mg to about 5 mg of astaxanthin.

15. The edible soft dental chew of any one of claims 9-13, wherein the outer layer comprises from about 0.3 mg to about 4 mg of astaxanthin.

16. The edible soft dental chew of any one of claims 9-15, wherein the outer layer includes an exterior surface facing away from the core, and wherein the exterior surface is substantially free of any protrusions configured to remove dental plaque and/or dental calculus from teeth of a subject.

17. The edible soft dental chew of any one of claims 9-16, wherein the core comprises a hardness reducing ingredient.

18. The edible soft dental chew of any one of claims 9-17, wherein the outer layer comprises a hardness reducing ingredient.

19. The edible soft dental chew of claim 17 or 18, wherein the hardness reducing ingredient is meat.

20. The edible soft chew of claim 19, wherein the meat is chicken meat.

21. The edible soft chew of claim 20, wherein the chicken meat is chicken meal.

22. The edible soft chew of claim 20 or 21, wherein the chicken meat is present in an amount of from about 5 wt % to about 10 wt %, by percent of the weight of the edible soft chew.

23. The edible soft chew of any one of claims 1-22, wherein the edible body has a disc shape.

24. A method of preventing or inhibiting dental plaque accumulation and/or dental calculus formation in a subject in need thereof, comprising administering to the subject at least one edible soft dental chew at least once per day, the edible soft dental chew comprising:
an edible body comprising astaxanthin,
wherein the edible body has an average Shore A hardness of less than about 50, and wherein the subject is a non-human animal.

25. The method of claim 24, wherein the edible body further comprises:
a core; and
an outer layer disposed on the core,
wherein at least one of the core and the outer layer comprises the astaxanthin, and wherein the outer layer has an average Shore A hardness of less than about 50.

26. The method of claim 25, wherein the core has an average Shore A hardness of less than about 50.

27. The method of any one of claims 24-26, wherein the outer layer comprises from about 0.01 mg to about 10.0 mg of astaxanthin.

28. The method of any one of claims 24-27, wherein the non-human animal has a weight of up to about 8 lbs, wherein the outer layer comprises from about 0.2 mg to about 0.8 mg of astaxanthin, and wherein the subject is administered at least one edible soft dental chew at least once per day.

29. The method of claim 28, wherein the subject is administered one edible soft dental chew twice per day.

30. The method of any one of claims 24-27, wherein the non-human animal has a weight of from about 8 lbs to about 20 lbs, wherein the outer layer comprises from about 1.8 mg to about 2 mg of astaxanthin, and wherein the subject is administered at least one edible soft dental chew at least once per day.

31. The method of claim 30, wherein the subject is administered two edible soft dental chews twice per day.

32. The method of any one of claims 24-27, wherein the non-human animal has a weight of from about 20 lbs to about 40 lbs, wherein the outer layer comprises from about 2.6 mg to about 4 mg of astaxanthin, and wherein the subject is administered at least one edible soft dental chew at least once per day.

33. The method of claim 32, wherein the subject is administered one edible soft dental chew twice per day.

34. The method of any one of claims 24-27, wherein the non-human animal has a weight of greater than about 40 lbs, wherein the outer layer comprises about 4 mg of astaxanthin, and wherein the subject is administered at least one edible soft dental chew at least once per day.

35. The method of claim 34, wherein the subject is administered two edible soft dental chews twice per day.

36. The method of any one of claims 24-35, wherein the non-human animal is at least 12 weeks old, and wherein the non-human animal is a canine or a feline.

37. The method of any one of claims 24-36, wherein the non-human animal has one or more of missing teeth, deciduous teeth, and cracked teeth.

38. The method of any one of claims 24-37, wherein teeth of the non-human animal are cleaned prior to the administration of the at least one edible soft dental chew.

39. The method of any one of claims 24-38, wherein teeth of the non-human animal are substantially free of dental plaque prior to the administration of the at least one edible soft dental chew, and wherein teeth of the non-human animal are substantially free of dental calculus prior to the administration of the at least one edible soft dental chew.
